(19) United States
(12) Patent Application Publication
(10) Pub. No.: US 2022/0297062 A1
(43) Pub. Date: Sep. 22, 2022

(54) GASEOUS POLLUTION CONTROL DEVICES AND METHODS OF REMOVING GASEOUS POLLUTANTS FROM AIR

(71) Applicants: David J. Wood, Guelph (CA); Stephanie D. Roth, Guelph (CA); Scott A. Shayko, Toronto (CA); William Van Heyst, Kitchener (CA); Xin Qiu, Mississauga (CA)

(72) Inventors: David J. Wood, Guelph (CA); Stephanie D. Roth, Guelph (CA); Scott A. Shayko, Toronto (CA); William Van Heyst, Kitchener (CA); Xin Qiu, Mississauga (CA)

(21) Appl. No.: 17/638,927

(22) PCT Filed: Aug. 28, 2020
(86) PCT No.: PCT/CA2020/051184

§ 371 Date: Feb. 28, 2022

(30) Foreign Application Priority Data
Aug. 30, 2019 (CA) 3053789

(51) Int. Cl. B01D 53/88; A61L 9/20; B01D 53/00; B01D 53/96

(52) U.S. Cl. CPC B01D 53/885; A61L 9/205; B01D 53/007; B01D 53/96; A61L 2209/12; A61L 2209/16; B01D 2257/404; B01D 2257/708; B01D 2257/7022; B01D 2259/802; B01D 2259/804; B01D 2255/802; B01D 2257/2064

(57) ABSTRACT

Gaseous pollution control devices and methods of removing pollutants from air are described herein. The devices include a body having a first end, a second end opposed to the first end, an upper wall and a lower wall opposed to the upper wall that all co-operate to define a cavity of the body. The device also includes one or more barriers within the body that form one or more channels within the body. At least one barrier has a flow disruptor to disrupt the flow of gas through the one or more channels. The device also includes a light source arranged within the body to direct light into the one or more channels. At least a portion of an inner surface of the device is at least partially coated with a photocatalytic composite material and the light source is configured to illuminate the coated inner surface to activate the photocatalytic composite material to remove the gaseous pollutants.

### RELATED APPLICATIONS

This application claims the benefit of Canadian Patent Application No. 3,053,789 entitled “Gaseous Pollution Control Devices and Methods of Removing Gaseous Pollutants from Air” filed Aug. 30, 2019. The entire contents of this application is hereby incorporated by reference herein in its entirety for all purposes.

### TECHNICAL FIELD

The embodiments disclosed herein relate to pollution control devices and, in particular, to gaseous PCDs for removing gaseous pollutants.

### INTRODUCTION

In recent years there has been considerable effort devoted to developing new technologies that solve ecological and environmental problems such as air pollution.

Common sources of air pollution include internal combustion engines, industrial plants, utility boilers, gas turbines, and commercial establishments such as service stations and dry cleaners. The types of air pollutants generated by these sources of pollution include, but are not limited, to particulate emissions such as coal ash, and gaseous pollutants including: sulphur compounds (such as SO₂ and SO₃), carbon monoxide, ozone, volatile organic compounds (VOCs) (such as ethylene gas), chlorinated solvents (such as trichloroethylene) and nitrogen oxides (commonly referred to collectively as “NOₓ”). Unless the air pollutants are treated prior to their release to their environment, these sources of air pollution will continue to contribute to the degradation of the environment or health risks of exposed populations.

Of the gaseous emissions listed above, the sources of VOC emissions are numerous. For example, VOCs are emitted from automobiles, petroleum refineries, chemical plants, dry cleaners, gas stations, and industrial facilities, among others.

NOₓ, typically used to refer to nitrogen (II) oxide (NO) and nitrogen (IV) oxide (NO₂), is primarily emitted from internal and external combustion sources, such as stationary power plants and automobile engines, and is particularly harmful to the environment.

Although traditional techniques such as physical adsorption, biofiltration, and thermal catalysis methods can remove substances such as, but not limited to, NOR, VOCs, ethylene gas, and chlorinated solvents from industrial emissions, they are not economically feasible for the removal of NOR, or other pollutants, at parts per billion (ppb) levels, which is desired, particularly for gaseous pollution control devices used in indoor environments.

Photocatalytic reactions offer potential for the removal of substances such as but not limited to NOR, VOCs, ethylene gas, and chlorinated solvents at parts per billion (ppb) levels in indoor environments. Upon illumination with light, photocatalysts release highly reactive photo-generated electron/hole pairs that can degrade surface-adsorbed species. Photocatalytic reactions do not consume extra chemicals or energy except for light energy, such as sunlight; as a result, they are widely considered the “greenest” method in combating gaseous pollutants.

Accordingly, there is a need for new or improved gaseous pollution control devices for removing gaseous pollutants, particularly from indoor environments.

### SUMMARY

According to some embodiments, a gaseous pollution control device is described herein. The device includes a body having: a first end and a second end opposed to the first end; and an upper wall and a lower wall opposed to the upper wall. The upper and lower walls extend between the first end and the second end and co-operate to define a cavity of the body. The cavity is configured to provide for gas to flow between the first end of the body and the second end of the body. The device also includes one or more barriers disposed within the cavity of the body to form one or more channels extending between the first end and the second end. At least one barrier has a flow disruptor to disrupt the flow of gas through the one or more channels. The device also includes a light source arranged within the body to direct light into the one or more channels. At least a portion of an inner surface of the pollution control device is at least partially coated with a photocatalytic composite material for removing a gaseous pollutant in the gas and the light source is configured to illuminate the inner surface at least partially coated with the photocatalytic composite material to activate the photocatalytic composite material to remove the gaseous pollutant in the gas.

According to some embodiments, the one or more barriers extend between the first end and the second end of the body.

According to some embodiments, the one or more barriers extend from the first end to the second end of the body.

According to some embodiments, the one or more barriers are coupled to the upper wall and the lower wall of the body to define the cavity.

According to some embodiments, the one or more barriers include more than one barrier, and each barrier is parallel with each other barrier.

According to some embodiments, the barriers are equally spaced from each other.

According to some embodiments, the barriers are unequally spaced from each other.

According to some embodiments, the flow disruptor extends from the barrier into one of the channels.

According to some embodiments, the one or more barriers include two barriers, each barrier having a flow disruptor extending into a common channel.

According to some embodiments, the one or more barriers include two barriers, each barrier having a plurality of a flow disruptors extending into a common channel, the plurality of flow disruptors being equally spaced or unequally spaced from each other along a length the channel.

According to some embodiments, the light source is positioned on a top surface or a bottom surface of the body and directed towards the one or more channels.

According to some embodiments, the light source is positioned on the one or more barriers and directed towards the one or more channels.

According to some embodiments, the light source is positioned on the flow disruptor and directed towards the one or more channels.

According to some embodiments, the light source is positioned at one of the first end and the second end and directed towards the one or more channels.

According to some embodiments, the barrier includes a plurality of flow disruptors dispersed along a length of the one or more channels and the light source is positioned between two of the plurality of flow disruptors.

According to some embodiments, the flow disruptor is coated with the photocatalytic composite material.

According to some embodiments, at least a portion of the one or more barriers is coated with the photocatalytic composite material.

According to some embodiments, the photocatalytic composite material is configured to remove NOR, volatile organic compounds, chlorinated solvents and/or ethylene (C₂H₄).

According to some embodiments, the NOₓ is NO or NO₂.

According to some embodiments, at least one of the first end and the second end of the body is configured to couple to a neighboring pollution control device.

According to some embodiments, the body is sized and shaped to be housed in an air duct.

According to some embodiments, the device also includes a cooling system.

According to some embodiments, the cooling system comprises a cooling apparatus disposed on or adjacent to an exterior surface of the body.

According to some embodiments, the cooling apparatus includes a water pump and a water pipe fluidly coupled to the water pump, the water pump configured to move water through the water pipe across the exterior surface of the body.

According to some embodiments, the light source is an ultraviolet (UV) light source.

According to some embodiments, a method of removing a gaseous pollutant from air is described herein. The method includes activating a photocatalytic composite material disposed on a surface of a pollution control device by exposing the photocatalytic composite material to light, the activating of the photocatalytic composite material providing for the photocatalytic composite material to releasably adsorb the pollutants in the air; and directing the air over the surface with the photocatalytic composite material for the pollutants in the air to adsorb to the photocatalytic composite material disposed on the surface.

According to some embodiments, the method also includes exposing the photocatalytic composite material to light after the photocatalytic composite material has removed the pollutant adsorbed thereto to release the pollutant from the photocatalytic composite material or clean the surface of other contaminants.

According to some embodiments, the method also includes directing the air over the surface to carry the pollutants out of the pollution control device.

According to some embodiments, the photocatalytic composite material is configured to remove NOₓ, volatile organic compounds, chlorinated solvents and/or ethylene (C₂H₄).

Other aspects and features will become apparent, to those ordinarily skilled in the art, upon review of the following description of some exemplary embodiments.

### BRIEF DESCRIPTION OF THE DRAWINGS

The drawings included herewith are for illustrating various examples of articles, methods, and apparatuses of the present specification. In the drawings:

FIG. 1 is a front, left-side perspective view of a pollution control device, according to one embodiment;

FIG. 2 is a front, right-side perspective view of the pollution control device shown in FIG. 1;

FIG. 3 is a front planar view of the pollution control device shown in FIG. 1;

FIG. 4 is a magnified planar view of the pollution control device shown in FIG. 1 showing UV lights positioned therein;

FIG. 5 is a side view of the pollution control device shown in FIG. 1;

FIG. 6 is a top view of the pollution control device shown in FIG. 1;

FIG. 7 is a top view of three of the pollution control device shown in FIG. 1 connected in series, according to one embodiment;

FIG. 8 is a schematic drawing of a magnified top down view showing channels and flow deflectors extending into the channels of a pollution control device, according to one embodiment;

FIG. 9 is a plot showing NO concentration over time for various UV light source intensities for various substrates the photocatalyst was applied to during experimentation. This was a stage of product development;

FIG. 10 is a plot showing NO concentration over time for various UV light sources on a standard acrylic substrate. This was part of the product development to determine whether light source plays an impact on the photocatalytic reduction performance;

FIG. 11 is a plot showing NO concentration over time for various substrates the photocatalyst was applied to during experimentation. This was done during product development to determine the best orientation (with respect to gas flow) and substrate to use in the pollution control;

FIG. 12 is a plot showing NO concentration over time for various substrate materials from Hollingsworth & Vose. This was done for experimentation with the PCD when the substrate was aligned perpendicular to gas flow, and thus the gas travelled through the porous substrate;

FIG. 13 is a plot showing NO concentration over time for various UV light sources and whether the substrates were illuminated from the front, back, or both sides;

FIG. 14 is a plot showing NO concentration over time when two substrates, oriented perpendicular to flow, were assembled in series;

FIG. 15 is a plot showing reduction ratio over time of a substrate oriented perpendicular to flow with varying flow rates;

FIG. 16 is a plot showing pressure loss versus flow rate of substrates, oriented perpendicular to flow, when substrates are assembled in series with one another;

FIG. 17 is a top-down view of three gaseous PCD, with substrates oriented perpendicular to air flow direction, coupled in series, according to another embodiment; and

FIG. 18 is a schematic diagram of two gaseous PCDs, with substrates oriented parallel to air flow direction, coupled in series, according to one embodiment.

### DETAILED DESCRIPTION

Various apparatuses or processes will be described below to provide an example of each claimed embodiment. No embodiment described below limits any claimed embodiment and any claimed embodiment may cover processes or apparatuses that differ from those described below. The claimed embodiments are not limited to apparatuses or processes having all of the features of any one apparatus or process described below or to features common to multiple or all of the apparatuses described below.

The term “ultraviolet (UV) light” as used herein refers to ultraviolet light with a wavelength of about 200 nm to about 400 nm. Not all wavelengths in this range need to be present in the “UV light” for the decomposition of the gaseous pollutants by the photocatalytic composite material.

The term “photocatalytic” as used herein, refers to the ability of a composite material of the disclosure to absorb light energy (UV and visible light) to remove gaseous pollutants, such as but not limited to nitrogen oxides and/or VOCs, to less harmful by-products, such as N₂.

The term “NOₓ” as used herein, refers to one, or a mixture of two or more nitrogen oxides, including NO, and NO₂, and the like formed, for example, during typical combustion processes.

The term “VOCs” as used herein, refers to one, or a mixture of two or more volatile organic compounds, and the like formed, for example, during natural or anthropogenic processes.

The term “chlorinated solvents” as used herein, refers to one, or a mixture of two or more chlorinated solvent compounds, such as trichloroethylene, and the like that may be formed or evolved from liquid products such as degreasers, paints, etc.

The terms “flow disruptor” or “vortex generator (VG)” may be used interchangeably and include any physical feature or character that disrupts (i.e. causes a disturbance in or induces turbulence in) a flow of gas thereacross. Disrupting a flow of gas may increase contact time, increase turbulence, trip flow, enhance mixing within an air stream, maintaining surface contact, preventing areas of recirculating flow, enhancing diffusion and mass transport, enhancing convection and heat transfer, etc. These physical features may take the form of changes to the surface morphology, such as adding roughness or dimples or curvature to a surface, or they may be physical additions to a surface, such as but not limited to adding a protrusion such as delta wings, prisms, etc.

Unless otherwise indicated, the definitions and embodiments described in this and other sections are intended to be applicable to all embodiments and aspects of the present application herein described for which they are suitable as would be understood by a person skilled in the art.

In understanding the scope of the present application, the term “comprising” and its derivatives, as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “having” and their derivatives. The term “consisting” and its derivatives, as used herein, are intended to be closed terms that specify the presence of the stated features, elements, components, groups, integers, and/or steps, but exclude the presence of other unstated features, elements, components, groups, integers and/or steps. The term “consisting essentially of”, as used herein, is intended to specify the presence of the stated features, elements, components, groups, integers, and/or steps as well as those that do not materially affect the basic and novel characteristic(s) of features, elements, components, groups, integers, and/or steps.

Terms of degree such as “substantially”, “about” and “approximately” as used herein mean a reasonable amount of deviation of the modified term such that the end result is not significantly changed. These terms of degree should be construed as including a deviation of at least ±5% of the modified term if this deviation would not negate the meaning of the word it modifies.

The term “and/or” as used herein means that the listed items are present, or used, individually or in combination. In effect, this term means that “at least one of” or “one or more” of the listed items is used or present.

As used in this application, the singular forms “a”, “an” and “the” include plural references unless the content clearly dictates otherwise. For example, an embodiment including “a compound” should be understood to present certain aspects with one compound or two or more additional compounds.

The present disclosure relates to gaseous pollution control devices (PCDs) for removing gaseous pollutants from air. In some embodiments, the PCDs described herein can be referred to as modular PCDs, such that the PCDs can have standardized units and be configured to be coupled to or used together with neighboring PCDs to remove gaseous pollutants.

In some embodiments, the gaseous PCDs described herein may include a main body or a frame that is sized or shaped to fit within an air duct within a building for removing pollutants from the air within a building. The frame may include positions for more than one PCD module. For example, there may be instances where PCD modules may be assembled lengthwise, beside each other, or stacked on top of each other, such as to fit an existing duct or body where the PCDs are to be installed.

However, the gaseous PCDs described herein should not be limited to being used to remove gases passing through air ducts in a building. Rather, the gaseous PCDs described herein may be used in any environment where gaseous PCDs are used, and in situations where no current air filtration or gaseous pollution control measures currently exist. Examples of such applications include, but are not limited to outdoor environments, automobiles, portable air handling units, naturally ventilated buildings (ex. Greenhouse), and other buildings without a forced HVAC system.

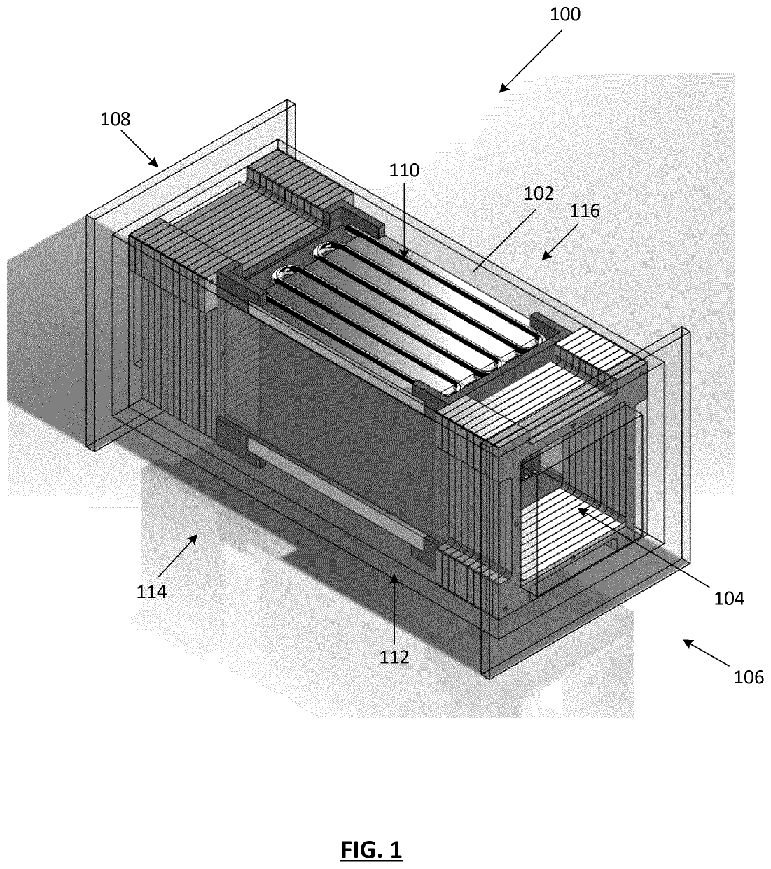

Referring now to FIG. 1, illustrated therein is a front, left-side perspective view of a pollution control device 100, according to one embodiment. Device 100 includes a body or frame 102 defining a cavity 104 that is configured to provide for air to flow therethrough.

Frame 102 has a first end 106 opposed to and spaced apart from a second end 108. In the embodiment shown in the figures body 102 is configured at the first end 106 and at the second end 108 to couple with an adjacent body of a neighboring pollution control device.

In the embodiment shown in the figures, body 102 has an upper wall 110, a lower wall 112, a first side wall 114 and a second side wall 116. Upper wall 110 is opposed to lower wall 112 and first side wall 114 is opposed to second side wall 116. Each of the upper wall 110, lower wall 112, first side wall 114 and second side wall 116 extend between the first end 106 and the second end 108 and co-operate to define the cavity 104.

Herein, the walls 110, 112 may be solid walls (i.e. have a continuous planar surface) or may be discontinuous walls (i.e. may include holes, slots or other discontinuities).

In the embodiments shown in the figures, body 102 is shown with a rectangular shape, but it should be noted that body 102 can have any appropriate shape for defining a cavity 104 that provides for air to pass through the body 102. For instance, body 102 may be configured to have a circular shape, in which case upper wall 110 and lower wall 112 may be continuous with each other.

Frame 102 may be constructed of any appropriate material for supporting the contents of the frame 102 (described in greater detail below).

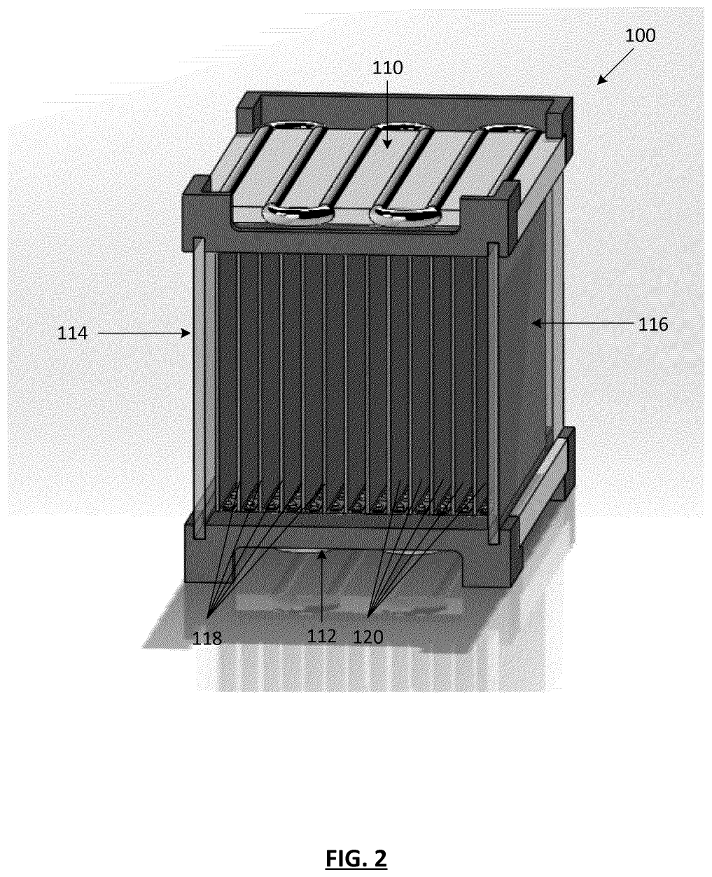

Device 100 includes at least one or more barriers 118 disposed within the cavity 104 of the body 102. Referring now to FIG. 2, illustrated therein is a front planar view of the device 100 of FIG. 1 showing a plurality of barriers 118. Each of the barriers 118 in the embodiment shown in FIG. 1 extends from the first end 104 of the body 102 to the second end 108 of the body 102, but it should be understood that each of the barriers 118 may only extend partially between first end 104 of the body 102 and the second end 108 of the body 102. The one or more barriers 118 co-operate with each other (or one of the side walls 114, 116), an interior surface of the upper wall 110 and/or an interior surface of the lower wall 112 to define one or more channels 120 that extend between the first end 106 of the body 102 and the second end 108 of the body 102. Again, in the embodiment shown in FIG. 1, the one or more channels 120 extend from the first end 106 of the body 102 to the second end 108 of the body 102. A gas (e.g. air) can therefore flow from the first end 106 of the body 102 to the second end 108 of the body 102 through the one or more channels 120.

In some embodiments, the one or more barriers 118 each extend between an interior surface of the upper wall 110 and an interior surface of the lower wall 112 of the device 100. For instance, the barriers 118 may be coupled to one or both of the interior surface of the upper wall 110 and the interior surface of the lower wall 112. In embodiments where the device 100 includes more than one barrier 118, the barriers 118 may be parallel with each other and may be equally spaced from each other.

As noted above, the barriers 118 may co-operate with an interior surface of the upper wall 110 and/or an interior surface of the lower wall 112 to define one or more channels 120. The one or more channels 120, depending on the position of the barriers 118, may have same dimensions or may have differing dimensions. In the embodiments shown in the figures, the channels 120 each have a same height, width and length. In some embodiments, the channels 120 may have a width in a range of about 5 mm to about 100 mm, or in a range of about 10 mm to about 50 mm, or be about 20 mm. The dimensions of the channels may vary depending on factors such as but not limited to the light source used, the application of the device 100 and the velocity of the gas passing through the channels 120.

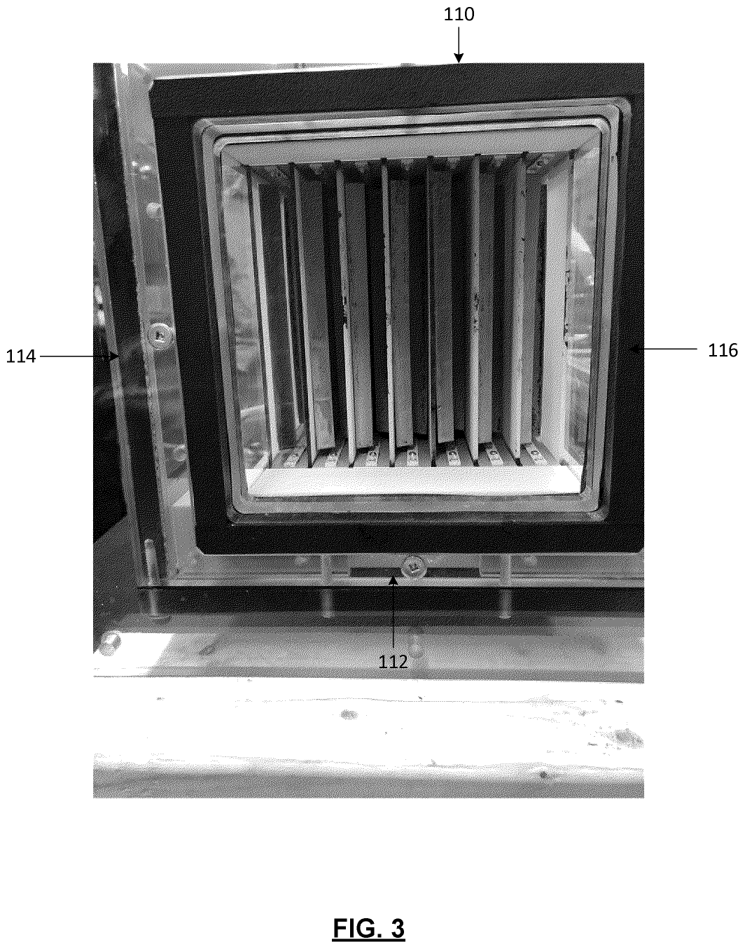

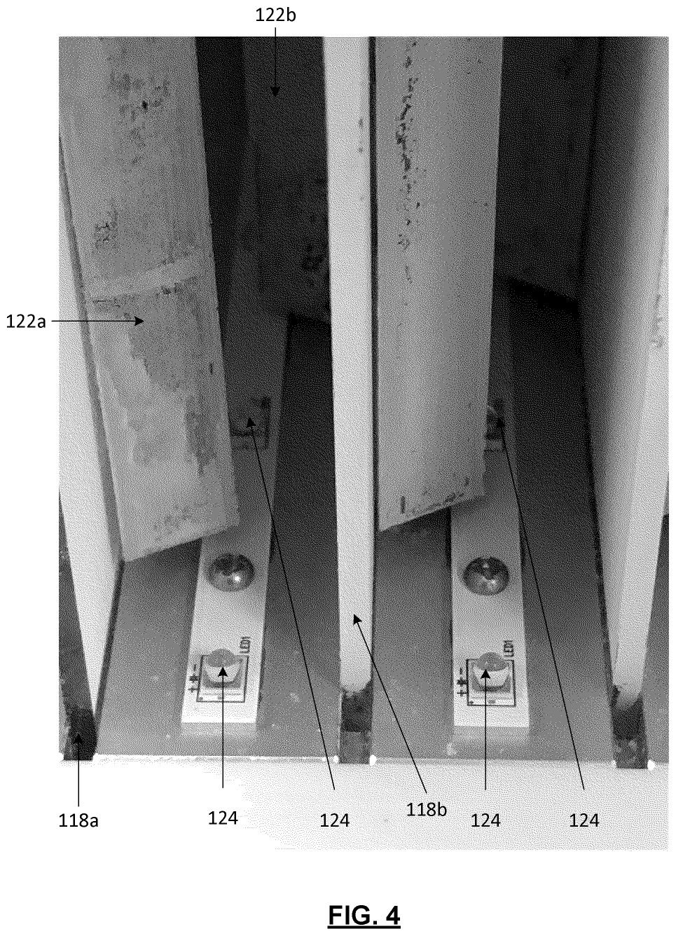

Each channel 120 includes at least one flow disruptor (or vortex generator) 122 (see FIG. 4). Referring now to FIG. 3, illustrated therein is another front planar view of the device 100 of FIG. 1 showing a plurality of barriers 118 and a flow disruptor 122 in each channel 120 defined by the barriers 118, according to one embodiment.

In the embodiment shown, each of the flow disruptors 122 is coupled to the one or more of the barriers 118 and extends into one of the channels 120. The flow disruptors 122 disrupt the flow of gas (e.g. air) through the device 100. For instance, the gas passing through the device 100 may have a turbulent flow after passing over a flow disruptor 122. The flow disruptors 122 generally increase a contact time between the air passing through the device 100 and the barriers 118 and/or the flow disruptors 122.

Each barrier 118 is generally coupled to a plurality of flow disruptors 122 that are spaced apart from each other between the first end 106 and the second end 108. In some embodiments, the flow disruptors 122 are arranged to extend inwardly to a respective channel 120 from either side of the channel 120. For instance, as shown in FIG. 4, each channel 120 can be defined by a first barrier 118a and a second barrier 118b spaced apart from the first barrier 118a. A first flow disruptor 122a can extend inwardly into the channel 120 from the first barrier 118a and a second flow disruptor 122b, from the second barrier, spaced apart from the first flow disruptor 122a in a direction of flow of the air through the device 100. Other configurations of flow disruptors may include surface dimpling or roughening, curvature of a barrier 118, individual prisms (such as delta wings), or the like.

In some embodiments, the flow disruptors 122 are oriented parallel to a direction of travel of the gas moving through the device 100. In other embodiments, the flow disruptors 122 are oriented to be perpendicular to a direction of travel of the gas moving though the device 100. In some embodiments, the flow disruptors 122 may not have any defined orientation relative to the direction of travel of the gas moving through the device 100.

In some embodiments, the flow disruptors 122 extend about halfway into the channels 120. For instance, in some embodiments, the channels 120 may have a width of about 20 mm and the flow disruptors may extend about 10 mm into the channels 120.

In some embodiments, the flow disruptors 122 are evenly dispersed along a length of the channels 120. For instance, in some embodiments, the flow disruptors may be spaced by a distance of about 5 mm to about 100 mm along the length of the channels 120, by about 30 mm to about 75 mm, or by about 60 mm.

In at least one embodiment, at least one inner surface of the channels 120 is at least partially covered with a photocatalytic composite material for removing gaseous pollutants in the air as the air passes through the channels 120. For instance, at least a portion of the flow disruptors 122 may be coated with a photocatalytic composite material for releasably binding and removing gaseous pollutants in the air. In some embodiments, at least a portion of the barriers 118 may be coated with the photocatalytic composite material.

The photocatalytic composite may be any composite material described in PCT/CA2016/051431, the contents of which are herein incorporated by reference. In some embodiments, the photocatalytic composite material is configured to remove NOₓ and/or volatile organic compounds. NOₓ may refer to NO or NO₂.

In at least one embodiment, at least one inner surface of the channels 120 is at least partially covered with a coating effective in killing bacteria and/or viruses. For instance, in at least one embodiment, the coating effective in killing bacteria and/or viruses may kill bacteria and/or viruses with an efficiency of less than about 90% deactivation, or about 90% deactivation, or greater than about 90% deactivation, or about 99% deactivation, or about 99.9% deactivation, or about 99.99% deactivation, or greater than about 99.99% deactivation, and in a timeframe of about 5 minutes, or less than about 5 minutes, or about 2 minutes, or less than about 2 minutes. In at least one embodiment, the coating effective in killing bacteria and/or viruses may have a photocatalytic component. In at least one embodiment, the coating effective in killing bacteria and/or viruses may be a contact-based disinfectant. For instance, in the light or in the dark, the coating effective in killing bacteria and/or viruses may be very effective at killing bacteria/virus that come in contact with it. In at least one embodiment, the coating effective in killing bacteria and/or viruses may be more effective in killing bacteria and/or viruses in the light. In at least one embodiment, the coating effective in killing bacteria and/or viruses may have a catalyst component that is bismuth based, such as but not limited to an oxide of bismuth.

In at least one embodiment, the coating effective in killing bacteria and/or viruses is known commercially as GermStopSQ™.

To remove NOₓ and/or volatile organic compounds, the photocatalytic composite material must be activated. Accordingly, gaseous PCD 100 includes a plurality of light sources 124 disposed within the channels 120 to activate the photocatalytic composite material. UV light sources 124 also act to release the NOₓ and/or volatile organic compounds from the photocatalytic composite material after they have been removed (and are therefore no longer harmful to the environment).

Light sources 124 are positioned on at least one inner surface of the device 100. For instance, light sources 124 may be positioned on inner surfaces of the device 100, such as on surfaces that neighbor the channels 120, and arranged to illuminate the photocatalytic composite material. The light sources 124 generate light and to direct light towards the photocatalytic composite material to activate the photocatalytic composite material to remove the gaseous pollutants in the air and to release the gaseous pollutants once they have been removed.

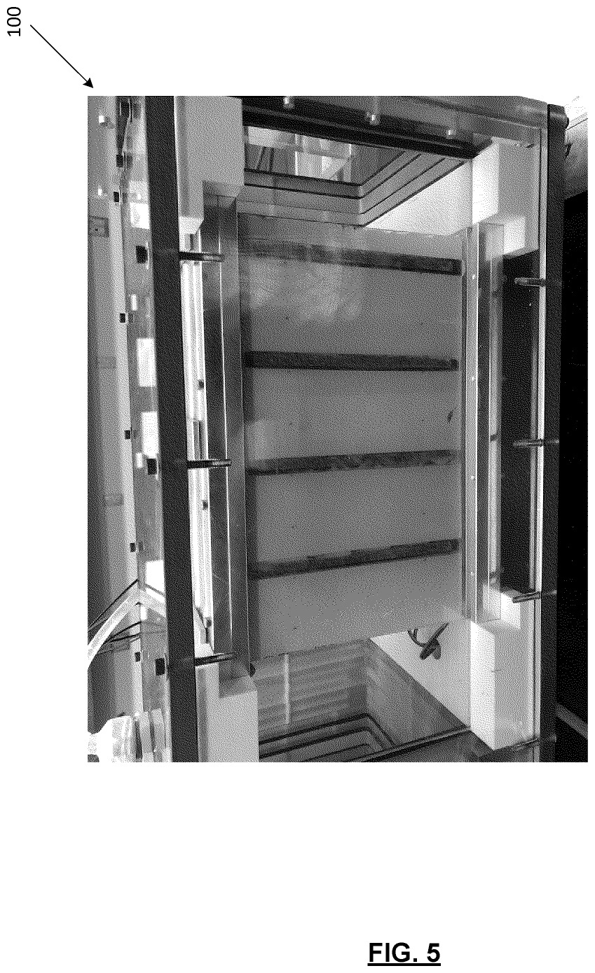

For instance, as shown in the embodiment in the figures, light sources 124 can be disposed on an interior surface of the upper wall 110 and/or on an interior surface of the lower wall 112. In some embodiments, the light sources 124 are positioned on both of the interior surface of the upper wall 110 and the interior surface of the lower wall 112. As shown in FIG. 5, the light sources 124 may be arranged on both of the interior surface of the upper wall 110 and the interior surface of the lower wall 112 and within the channel 120 such that light generated by the light sources 124 is directed into the channel 120 as air passes through the channel 120. In some embodiments, two light sources 124 can be arranged on both of the interior surface of the upper wall 110 and the interior surface of the lower wall 112 and within the channel 120 between adjacent flow disruptors 122 to remove NOₓ and/or volatile organic compounds. This arrangement can be repeated along the length of each of the channels 120 or can be repeated in neighboring modules positioned side-by-side. In this arrangement the light generated by the light sources 124 may have a narrow beam angle (e.g. in a range of about 55 degrees to about 130 degrees, or about 75 degrees).

In some embodiments, the one or more light sources 124 are UV light sources that emit light having a wavelength in a range of about from 10 nm to about 400 nm. In some embodiments, the light sources 124 emit light having a wavelength outside of the UV spectrum noted above. In some embodiments, the light sources 124 are LED UV light sources. The UV light sources may emit an LED radiant flux of in a range between about 0 W and 10 W, or between about 1 W and about 5 W, or be about 2 W.

In some embodiments, light sources 124 may be spaced apart by a distance in a range of about 0.5 cm to about 10 cm, or in a range of about 1 cm to about 5 cm, or of about 2 cm or of about 3 cm along a length of each channel 120.

In some embodiments, the device 100 is sized and shaped to be housed in an air duct. In other embodiments, the device 100 may be a stand-alone unit.

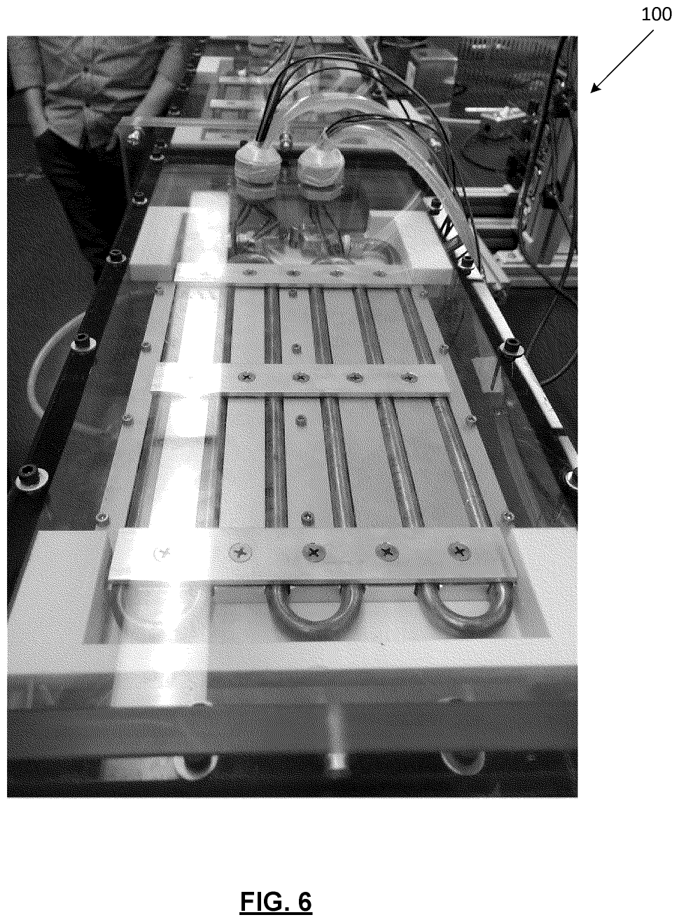

In some embodiments, the device 100 may include one or more cooling systems to remove heat from the device 100 (e.g. heat generated by the light sources 124). As shown in FIG. 6, the cooling system 130 may include one or more cooling apparatus 132. The cooling apparatus 132 may be disposed on or adjacent to an exterior surface of the device 100, such as but not limited to on at least one of an exterior surface of the upper wall 110 and an exterior surface of the lower wall 112 In other embodiments, the cooling system 130 may be positioned inside of the device 100.

The cooling apparatus 132 may include a water pump (not shown) and a water pipe 134 fluidly coupled to the water pump. The water pump may be configured for moving water through the water pipe 134 across the exterior surface of the at least one of the upper wall 110 and the lower wall 112 of the body 102.

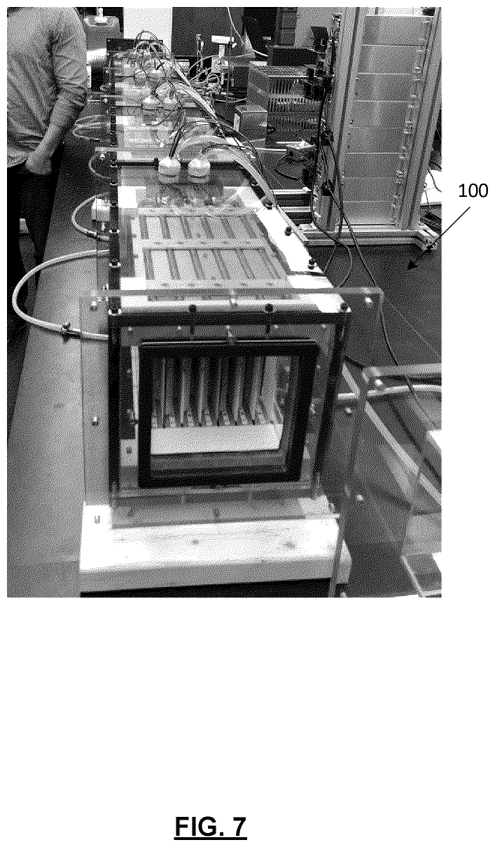

In some embodiments, at least one of the first end 106 and the second end 108 of the body 102 is configured to couple to a neighboring device 100. As shown in FIG. 7, at least two of the devices 100 described herein can be coupled to each other and operated in series. In some embodiments, three or more devices 100 described herein can be coupled to each other and operated in series.

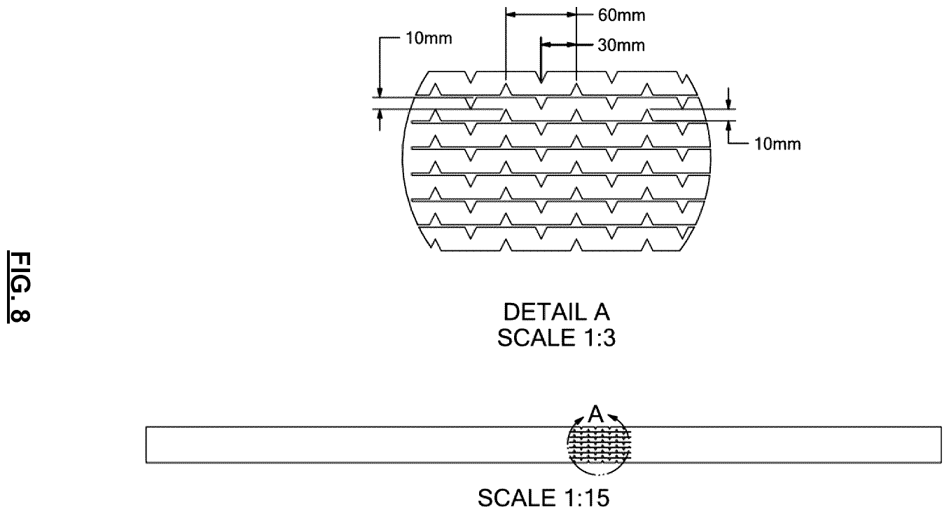

Referring to FIG. 8, illustrated therein is a schematic drawing of a top down view of one embodiment of a configuration of barriers 118 and flow disruptors 122 within a body 102. In this embodiment, the neighboring flow disruptors 122 are spaced apart from each other along each channel 120 by 60 mm, flow disruptors 122 on opposed barriers 118 and extending into a common channel 120 are spaced apart from each other along their common channel 120 by 30 mm, and each flow disruptor 122 extends into each channel 120 from a barrier 118 by a distance of 10 mm.

In some embodiments, a method of removing pollutants from gas is described herein. The methods described herein generally include activating a photocatalytic composite material disposed on a surface of a pollution control device by exposing the photocatalytic composite material to light and directing gas (e.g. air) over the surface with the photocatalytic composite material. Generally, the device is a pollution control device disclosed herein.

Directing the air over the photocatalytic composite material generally causes pollutants in the gas to adhere to the photocatalytic composite material. Once the pollutants in the gas adsorb to the photocatalytic composite material, the pollutants generally remove into a form that is not harmful to the environment and then are released from the surface.

In some embodiments, the methods also include removing the decomposed gaseous pollutants from the photocatalytic composite material by exposing the photocatalytic composite material to light (e.g. UV light)

In some embodiments, the methods also include directing air or gas over the photocatalytic composite material to remove the decomposed gaseous pollutants from the pollution control device.

While the above description provides examples of one or more apparatus, methods, or systems, it will be appreciated that other apparatus, methods, or systems may be within the scope of the claims as interpreted by one of skill in the art.

### EXAMPLES

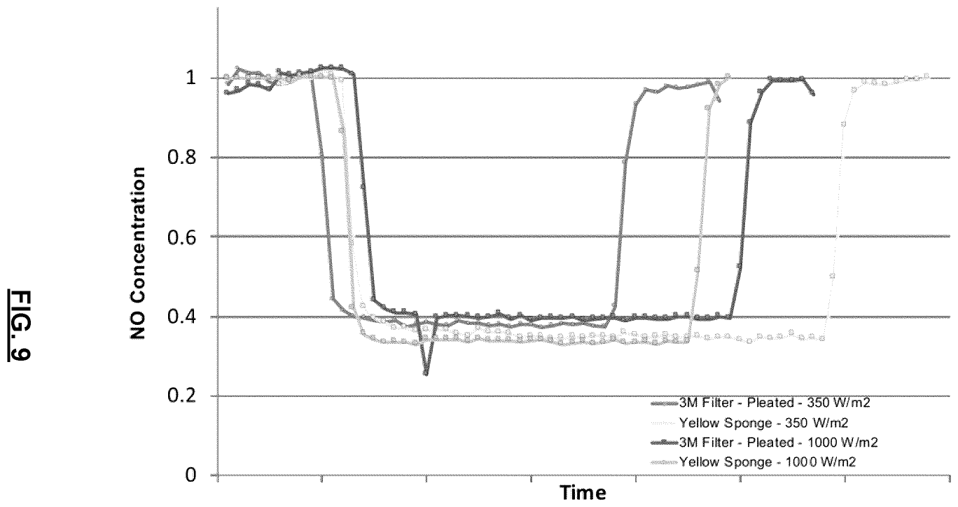

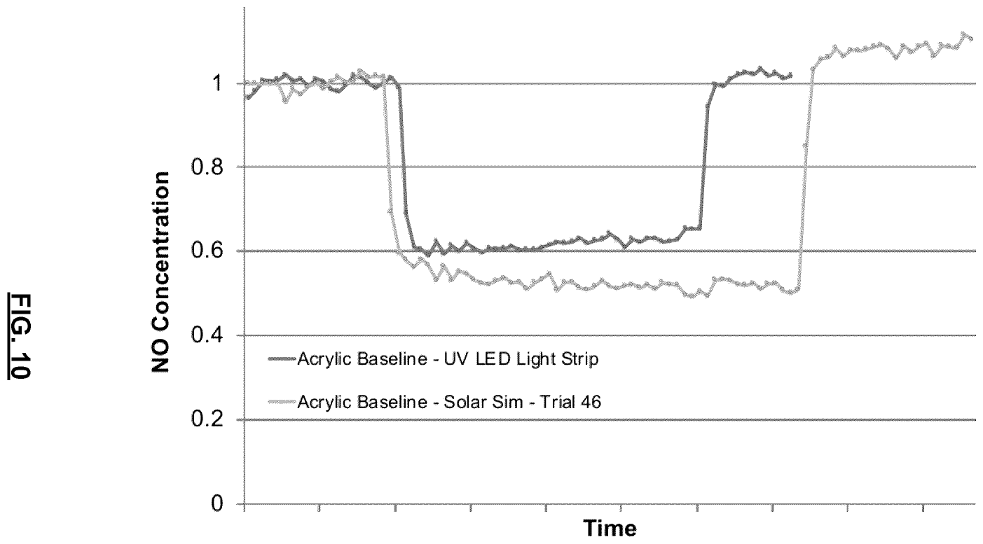

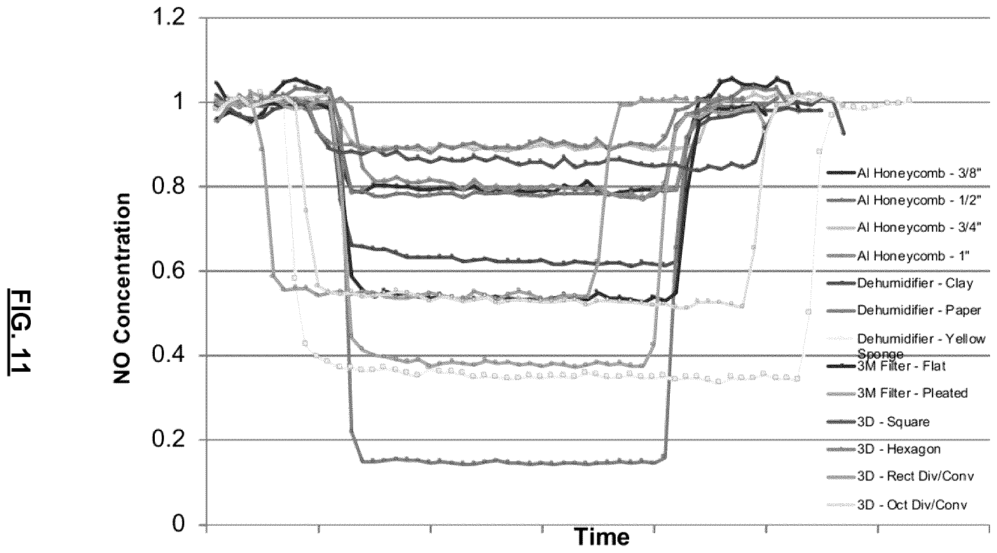

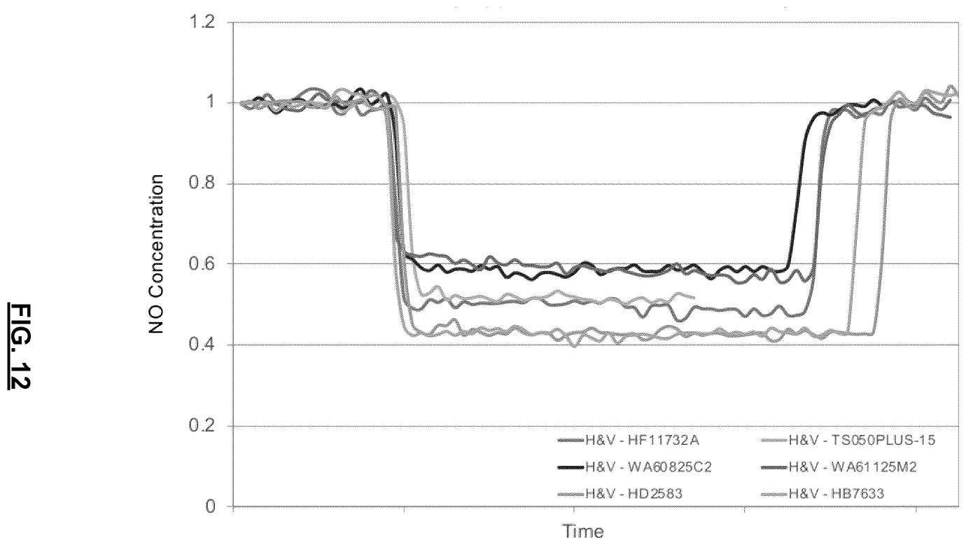

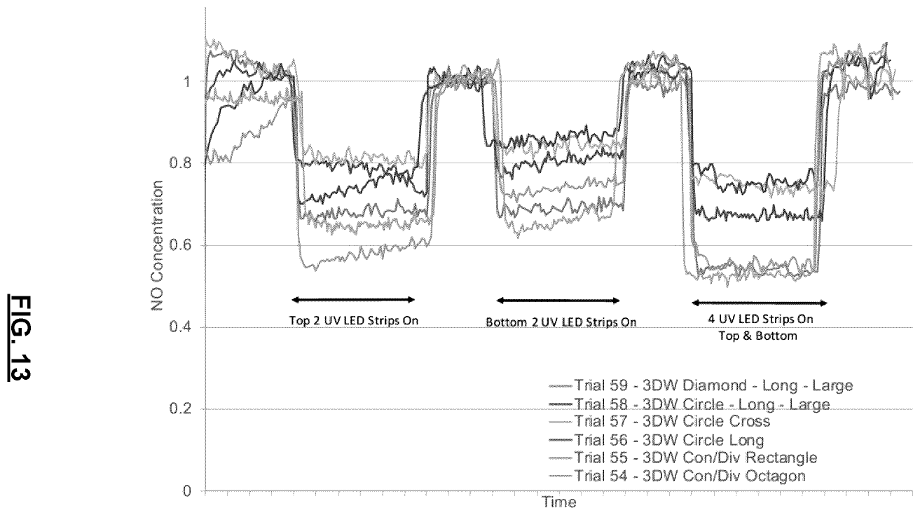

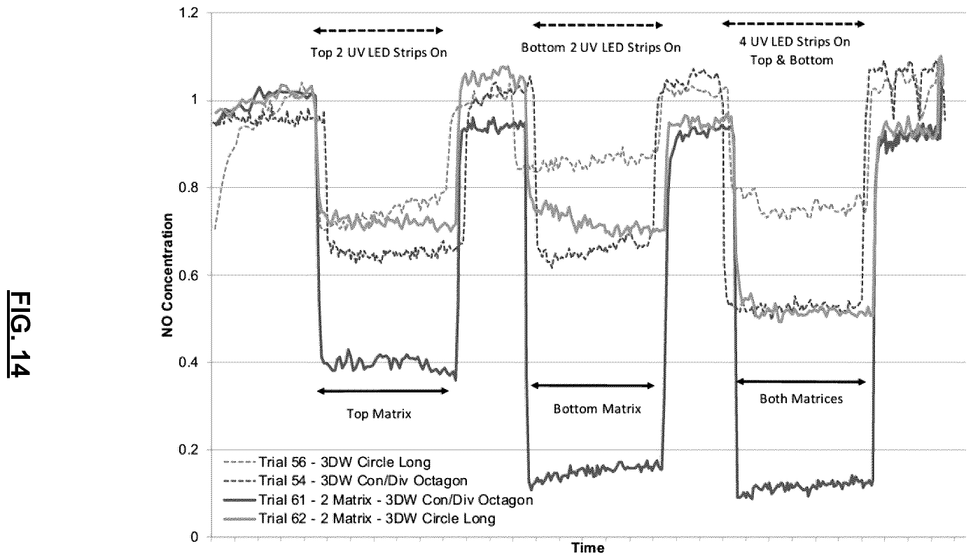

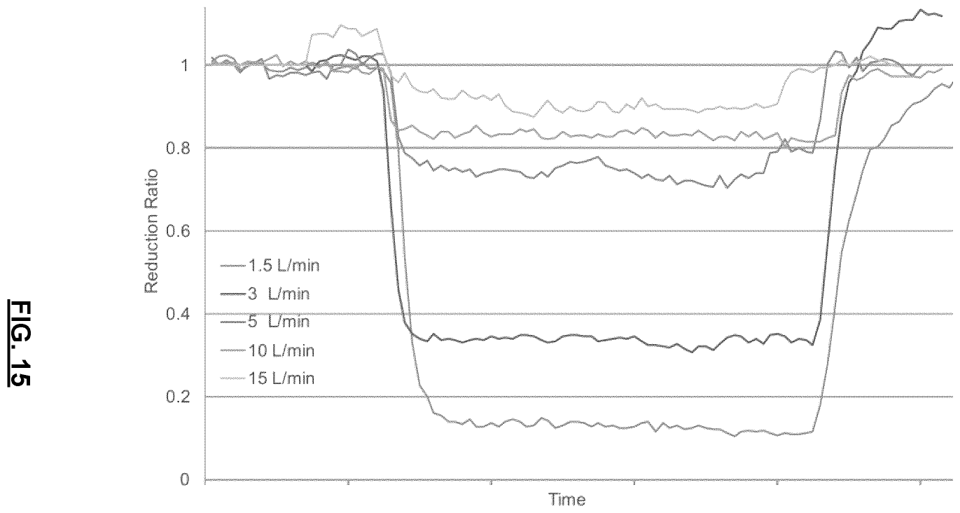

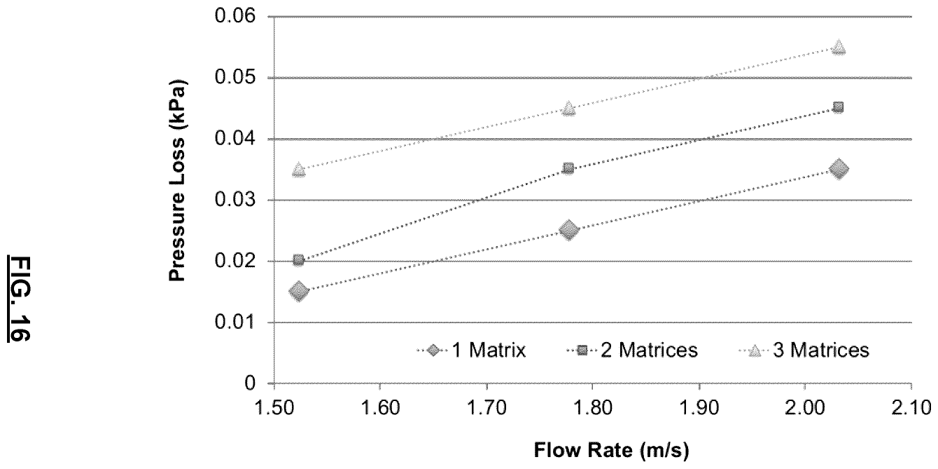

Trials for the NOₓ reduction potential of at least one of the embodiments of gaseous PCDs described herein were run with NO as the tracer gas, using laboratory air as the dilution gas. As a result, reduction potential was determined by finding the ratio of reduced NO concentration (when the system lights were activated) over the initial NO concentration (prior to the lights turning on). Some important conclusions have surfaced from the experiments conducted for at least one of the embodiments of gaseous PCDs described herein. These results include:
1. Intensity of lighting does not appear to have a significant impact on reduction efficiency, provided the entire surface is illuminated by light (see FIG. 9).
2. UV LED lighting can be as effective at activating the photocatalyst as the light produced from a solar simulator (see FIG. 10).
3. Surface area plays a significant role in reduction efficiency, as seen by the difference between the 3M ‘flat’ (stretched) vs. ‘pleated’ sample (see FIG. 11).
4. Pre-fabricated gaseous PCDs can provide excellent reduction potential but at a cost of adding significantly more pressure losses to the system (see FIGS. 11 and 12).
5. Surface orientation to lighting directly influences reduction potential. Honeycomb (e.g. porous substrate) gaseous PCDs whose cell surfaces ran parallel to each other and to the light source showed significantly less reduction efficiency than the converging and diverging channel configurations (see FIG. 11).
6. The custom 3D converging-diverging channel configurations had the highest reduction efficiency of all 3D printed configurations (see FIG. 11).
7. Highest reduction efficiencies are achieved when lighting was supplied both at the top and bottom of a sample. Lighting only from the top produced reduction efficiencies equal or better than when light was only provided from the bottom (see FIG. 13).
8. Higher reduction efficiencies are achieved by using two gaseous PCDs in series, as opposed to using a single gaseous PCD (see FIG. 14).
9. Reduction efficiency decreases significantly as the velocity through the system increases (see FIG. 15).
10. 3D converging-diverging octagon matrix had low pressure losses in the large-scale test chamber for systems containing up to three gaseous PCDs (see FIG. 16).

FIG. 9 shows NO Reduction Trial Results, where inlet concentration of NO was 50 ppb, using the Solar Simulator (Visible+UVA spectrum) to test the impact of light intensity. In both scenarios, the light illuminated the entire surface of the gaseous PCD. Higher intensity light did not produce a significant increase in reduction efficiency.

FIG. 10 shows NO Reduction Trial Results, where inlet concentration of NO was 50 ppb, using the Solar Simulator (Visible+UVA spectrum) and a UV LED strip lighting. There was only a small (˜10% difference) in the reduction efficiency between the light sources. This illustrates that the UV LED lights are providing sufficient illumination to activate the photocatalytic reduction reaction.

FIG. 11 shows NO Reduction Trial Results, where inlet concentration of NO was 50 ppb, using the Solar Simulator (Visible+UVA spectrum) for pre-fabricated and custom 3D printed IAPS gaseous PCDs. Pre-fabricated gaseous PCDs provide good reduction efficiencies, but will increase pressure losses through the gaseous PCD system. Of the custom 3D printed matrices, the converging and diverging configurations provided the best reduction efficiencies.

FIG. 12 shows NO Reduction Trial Results, where inlet concentration of NO was 50 ppb, using the Solar Simulator (Visible+UVA spectrum) for pre-fabricated porous material. All types of gaseous PCDs had relatively similar reduction potential (between 40-60%).

FIG. 13 shows NO Reduction Trial Results, where inlet concentration of NO was 50 ppb, using UV LED lighting. The top surface and the bottom surface each had two LED strip lights to illuminate the respective surfaces. Best reduction efficiency was achieved using both top and bottom illumination. When illuminated separately, top lighting provided equal or greater reduction efficiency than bottom lighting.

FIG. 14 shows NO Reduction Trial Results, where inlet concentration of NO was 50 ppb, using UV LED strip lighting. Graph illustrates that for both matrix geometries that the use of two gaseous PCDs (solid lines) provides better reduction efficiencies, than if the system used a single gaseous PCD, and that best reduction is achieved when matrices are illuminated from both top and bottom.

FIG. 15 shows NO Reduction Trial Results, where inlet concentration of NO was 50 ppb, using UV LED strip lighting on the octagonal converging/diverging matrix. Flow rate through the test chamber is shown to have a significant impact on the reduction efficiency of the IAPS.

FIG. 16 shows pressure losses recorded in the large scale test chamber. Trials were run at different flow rates for either one, two or three octagonal converging/diverging matrices in series. Overall the maximum pressure losses anticipated for the system were approximately 0.055 kPa.

### Gaseous PCD Initial Prototype Design Based on Small Scale Test Chamber Results

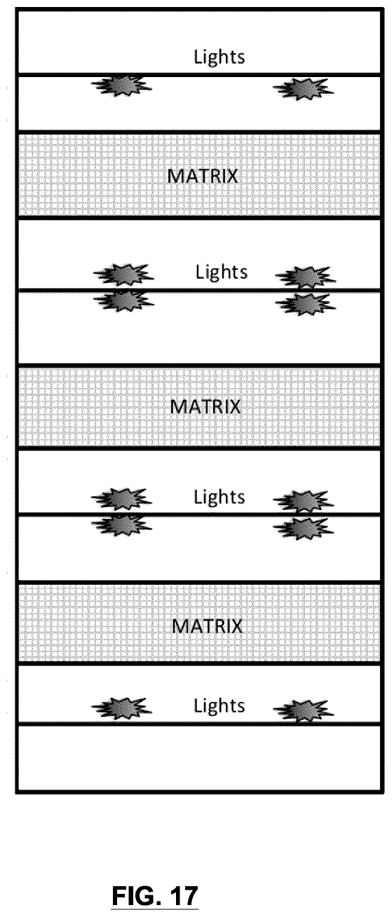

Tests completed in the Small Scale Test Chamber allowed for the initial prototype design of the Smog Stop Gaseous PCD, a simplified illustration of the system configuration is provided in FIG. 17. In order to achieve a high NO reduction efficiency of 80% or greater, at least two gaseous PCDs are required in series, with the gaseous PCDs oriented perpendicular to the gas stream such that the gas passes through the gaseous PCD. As the NO photocatalytic reduction reaction occurs on the surface of the gaseous PCD, the surface can become saturated and therefore will require time when the gaseous PCD is not activated. As a result, a third gaseous PCD will be required in series. The third gaseous PCD, in conjunction with programmed lighting controls, will allow one gaseous PCD to be off for surface desorption of contaminants while there will still be two gaseous PCDs on and activated to achieve the required reduction efficiency.

FIG. 17 shows the Gaseous PCD Initial Prototype Design configuration for residential and commercial HVAC ductwork. The system consists of three barriers in series, with the gaseous PCD oriented perpendicular to the gas stream such that the gas passes through the gaseous PCD, and lighting for the front and back of each barrier. This configuration, along with programed lighting controls, has been developed in order to achieve a high reduction efficiency, while allowing one barrier to be inactive and contaminants to desorb from its surface.

### Test Duct Results Summary

Due to the qualitative nature of many of the results obtained from the large scale Test Duct trials, results are summarized here in a bulleted list:
1. Tests were performed using:
   a. Porous fabric media
   b. Multiple gaseous PCDs in series, with the gas passing through the fabric gaseous PCD
   c. Lighting system was using the UV LED
2. Fabric Gaseous PCD tests, with gaseous PCDs oriented perpendicular to the gas stream, had results that indicated:
   a. Extremely large pressure losses with multiple gaseous PCDs in series, pressure losses were outside of the design criteria for the Gaseous PCD.
   b. Contact time of the gas passing through the fabric gaseous PCD is not sufficient to generate a sufficient reduction in pollutants, even with multiple gaseous PCDs in series
   c. UV LED lights obtained were difficult to wire, expensive and had very long lead times
3. Based on the results from gaseous PCDs oriented perpendicular to the gas stream, it was decided that new tests should be completed with the gaseous PCDs oriented parallel to the stream. This new case will allow the gas would flow along the gaseous PCD, with the hopes of increasing contact time with the photocatalyst.
4. Parallel Flow Tests were run with a coated fabric laminated to an acrylic plate (total length 25 cm) with:
   a. Fabric on one side of the plate and LED lights on the other
   b. Fabric on both sides of the plate with LED lights hung between plates
5. Parallel Flow and Fabric Laminated Barrier test results indicated:
   a. Better reduction when both sides of the plate are coated/laminated with fabric
   b. LEDs emit significant amounts of heat
   c. Assembly of the system with the LEDs placed between plates is difficult.
      i. Location of the LEDs should be moved to the top and bottom of the chamber for ease of construction, but distribution of light is required to be studied to ensure sufficient illumination of the plates
   d. Heat generation was enough to burn the fabric material and melt acrylic plates,
      i. Glass plates, or other material that can withstand higher temperatures, coated directly with the photocatalyst will be required for operation and testing
   e. A heat dissipation unit was designed to allow for heat removal in an enclosed system where heat is retained internal to the system
6. Parallel Flow and Glass Plate Gaseous PCD test results
   a. A single length of plates (25 cm for lab testing purposes) generates a measurable reduction of pollutants but this reduction is not sufficient to offer as a product and decreases as the gas velocity increases.
      i. Gaseous PCD units should be manufactured with a minimum plate length of 50 cm to offer clients a reasonable removal from a single stage of unit
      ii. Assembling multiple gaseous PCD units in series will allow for more contact and better reduction.
      iii. There may be ways of improving gas flow between plates to increase contact of the gas with the photocatalyst through the use of flow disruptors or vortex generators. CFD studies will be required to optimize fluid flow and contact time.
      iv. CFD studies should look at: plate spacing, gas turbulence and mixing, etc.

### Gaseous PCD Final Prototype Design

A final prototype design for the Gaseous PCD was developed based on the combination of the Small Scale and larger Test Duct testing and gaseous PCD design development. This design includes glass plates coated on both sides with the photocatalyst, plates oriented parallel to the gas stream, and LED lighting on the top and bottom of the system.

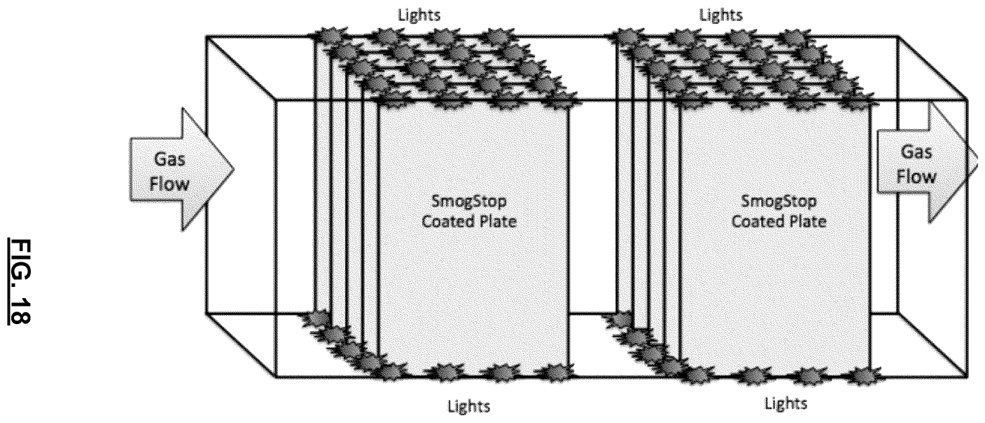

Gaseous PCD units are 50 cm modules and can be assembled in series, as illustrated in FIG. 18, to increase pollutant reduction by increasing the available contact area and, hence, contact time.

A visual illustration of another embodiment of the Gaseous PCD, illustrating two gaseous PCD units in series, is provided in FIG. 18.

### Gaseous PCD Prototype Design Optimization

Below are the steps that were taken in the CFD to evaluate the different design options.

Step 1: Evaluated different plate spacing and varying lengths of plates. These have no vortex generators (VGs) included. Air velocity was fixed at 2 ms⁻¹.

Findings: It was evident that longer plate lengths and higher plate densities were beneficial for performance.

| | # of Plates | | |
|---|---|---|---|
| (spacing) | 9 | 16 | 28 |
| Length (cm) | (2 cm) | (1 cm) | (0.5 cm) |

Percent Reduction of NO (%)

| | | | |
|---|---|---|---|
| 16 | 23.4 | 51.3 | 81.6 |
| 26 | 32.7 | 69.7 | 96.2 |
| 36 | 51.8 | 80.3 | 98.8 |
| 50 | 62.2 | 90.0 | — |

Step 2: Incorporated VGs into the analysis for select cases in step 1. VGs are only on one side. Velocity was 2 ms⁻¹.

Findings: The inclusion of VGs was beneficial for performance.

| | # of Plates | | |
|---|---|---|---|
| (spacing) | 9 | 16 | 28 |
| Length (cm) | (2 cm) | (1 cm) | (0.5 cm) |

Percent Reduction of NO (%)

| | | | |
|---|---|---|---|
| 16 | 23.4 | 51.3 | 81.6 |
| 16 cm + VGs | — | 76.7 | — |
| 26 | 32.7 | 69.7 | 96.2 |
| 26 cm + VGs | — | 93.8 | — |
| 36 | 51.8 | 80.3 | 98.8 |
| 50 | 62.2 | 90.0 | — |
| 50 cm + VGs | 90.1 | — | — |

Step 3: Incorporated VGs onto both sides of the channel. Velocity was 2 ms⁻¹.

Findings: Including VGs on both sides of the channel was identified as the best option. Abandoned 16 cm plate lengths and the 28 plate configuration.

| | # of Plates | | |
|---|---|---|---|
| (spacing) | 9 | 16 | 28 |
| Length (cm) | (2 cm) | (1 cm) | (0.5 cm) |

Percent Reduction of NO (%)

| | | | |
|---|---|---|---|
| 16 | 23.4 | 51.3 | 81.6 |
| 16 cm + VGs | — | — | — |
| 26 | 32.7 | 69.7 | 96.2 |
| 26 cm + VGs | 86.2 | 96.6 | — |
| 36 | 51.8 | 80.3 | 98.8 |
| 50 | 62.2 | 90.0 | — |
| 50 cm + VGs | 98.6 | 99.8 | — |

Step 4: Final step was to collect data on the latest configuration at 0.5, 1.0, and 2.0 ms⁻¹.

| | | Gaseous PCD Length (cm) | | | | | |
|---|---|---|---|---|---|---|---|
| | | 26 cm | | | 50 cm | | |
| Air Velocity (m/s) | | 0.5 | 2 | 3 | 0.5 | 2 | 3 |
| 2 cm | NO | 92.3 | 86.2 | 83 | 99.3 | 98.6 | 98.4 |
| Plate | Removal | | | | | | |
| Spacing | (%) | | | | | | |
| | Pressure | 10.3 | 176 | 406 | 21.0 | 367 | — |
| | Drop | | | | | | |
| | (Pa) | | | | | | |
| 1 cm | NO | 98.8 | 96.6 | 95.7 | 99.9 | 99.9 | 99.8 |
| Plate | Removal | | | | | | |
| Spacing | (%) | | | | | | |
| | Pressure | 9.5 | — | 325 | 18.6 | 271 | 677 |
| | Drop | | | | | | |
| | (Pa) | | | | | | |

The data presented below represents the current collection of data for the physical testing of the gaseous PCD. The gaseous PCD design used in this testing was the final configuration that was optimized using LightTools and Fluent.

The first table below summarizes the data from testing two configurations (1 cm and the 2 cm plate spacing). A comparison of the 50% blockage VG size in both configurations reveals that the performance is very similar. Given the material and lighting cost of the 1 cm spacing configuration is nearly double that of the other, this configuration was abandoned. Through this analysis the optimal VG size was determined to be 10 mm in the 2 cm plate spacing.

The second table below shows results from adding multiple gaseous PCDs in series. Clearly, as the number of gaseous PCDs increases so does the performance.

TABLE 1
Summary of single gaseous PCD testing

| | Single Gaseous PCD Testing | | | | | | | | | | | |
|---|---|---|---|---|---|---|---|---|---|---|---|---|
| | 1 cm Spacing | | | | | | 2 cm Spacing | | | | | |
| | Duct Air Velocity | | | | | | | | | | | |
| | 0.5 m/s | | 1.0 m/s | | 2.0 m/s | | 0.5 m/s | | 1.0 m/s | | 2.0 m/s | |
| LED Power | Percent Reductions (~100 ppb inlet concentration) | | | | | | | | | | | |
| Setting | NOₓ | NO | NOₓ | NO | NOₓ | NO | NOₓ | NO | NOₓ | NO | NOₓ | NO |
| VG | | | | | | | 75% blockage from 15 mm VG | | | | | |
| configuration | | | | | | | | | | | | |
| 1.0 W | | | | | | | 0% | 17% | 0% | 9% | | |
| 2.5 W | | | | | | | 12% | 41% | 0% | 13% | | |
| VG | 50% blockage from 5 mm VG | | | | | | 50% blockage from 10 mm VG | | | | | |
| configuration | | | | | | | | | | | | |
| 0.5 W | | | | | | | | | | | | |
| 1.0 W | 4.7% | 34.5% | 0% | 22% | 0% | 8.1% | 2% | 32% | 0% | 11.4% | 1.4% | 5.8% |
| 1.5 W | | | | | | | 6.4% | 28.5% | 0% | 12.7% | | |
| 2.0 W | | | | | | | 16% | 39.8% | 1.8% | 19.1% | | |
| 2.5 W | 22% | 54% | 0% | 29.7% | 0% | 13.5% | 19.8% | 57.5% | 0% | 22% | 0% | 8% |
| 2.5 W | | | | | | | 17% | 38% | 2% | 15% | | |
| RETEST | | | | | | | | | | | | |
| 2.5 W | | | | | | | 17% | 35% | | | | |
| RETEST | | | | | | | | | | | | |
| VG | | | | | | | 25% blockage from 5 mm VG | | | | | |
| configuration | | | | | | | | | | | | |
| 2.5 W | | | | | | | 20% | 33% | 5% | 16% | | |
| VG | | | | | | | 0% blockage (No VG) | | | | | |
| configuration | | | | | | | | | | | | |
| 2.5 W | | | | | | | 16%* | 37%* | 14% | 17% | 1.6% | 5% |
| 2.5 W | | | | | | | 13% | 20% | 6.6% | 12.6% | | |
| RETEST | | | | | | | | | | | | |

*Test was the first conducted in the series of NO VG testing. Most likely why it performed so well the first time.

TABLE 2
Summary of testing results from 1, 2, and 4 gaseous PCDs in series

| | 1 Gaseous PCD in Series NOx testing | | | | | |
|---|---|---|---|---|---|---|
| | 2 cm Spacing + 10 mm VG | | | | | |
| | Duct Air Velocity | | | | | |
| | 0.5 m/s | | 1.0 m/s | | 2.0 m/s | |
| LED Power | Percent Reductions | | | | | |
| Setting | NOₓ | NO | NOₓ | NO | NOₓ | NO |
| 2.5 W | 17% | 38% | 2% | 15% | 0% | 8% |
| | 2 Gaseous PCDs in Series NOx testing | | | | | |
| | 2 cm Spacing + 10 mm VG | | | | | |
| | Duct Air Velocity | | | | | |
| | 0.5 m/s | | 1.0 m/s | | 2.0 m/s | |
| LED Power | Percent Reductions | | | | | |
| Setting | NOₓ | NO | NOₓ | NO | NOₓ | NO |
| 2.5 W | 38% | 80% | 5.1% | 34% | 2.2% | 22% |
| | 4 Gaseous PCDs in Series NOx testing | | | | | |
| | 2 cm Spacing + 10 mm VG | | | | | |
| | Duct Air Velocity | | | | | |
| | 0.5 m/s | | 1.0 m/s | | 2.0 m/s | |
| LED Power | Percent Reductions | | | | | |
| Setting | NOₓ | NO | NOₓ | NO | NOₓ | NO |
| 2.5 W | 65% | 97% | 30% | 67% | 0% | 30% |

While the applicant's teachings described herein are in conjunction with various embodiments for illustrative purposes, it is not intended that the applicant's teachings be limited to such embodiments as the embodiments described herein are intended to be examples. On the contrary, the applicant's teachings described and illustrated herein encompass various alternatives, modifications, and equivalents, without departing from the embodiments described herein, the general scope of which is defined in the appended claims.

What is claimed is:

1. A gaseous pollution control device comprising:
a body having:
a first end and a second end opposed to the first end; and
an upper wall and a lower wall opposed to the upper wall, the upper and lower walls extending between the first end and the second end and co-operating to define a cavity of the body, the cavity being configured to provide for gas to flow between the first end of the body and the second end of the body;
one or more barriers disposed within the cavity of the body to form one or more channels extending between the first end and the second end; at least one barrier having a flow disruptor to disrupt the flow of gas through the one or more channels; and
a light source arranged within the body to direct light into the one or more channels;
wherein at least a portion of an inner surface of the pollution control device is at least partially coated with a photocatalytic composite material for removing a gaseous pollutant in the gas and the light source is configured to illuminate the inner surface at least partially coated with the photocatalytic composite material to activate the photocatalytic composite material to remove the gaseous pollutant in the gas.

2. The pollution control device of claim 1, wherein the one or more barriers extend between the first end and the second end of the body.

3. The pollution control device of claim 1 or claim 2, wherein the one or more barriers extend from the first end to the second end of the body.

4. The pollution control device of any one of claims 1 to 3, wherein the one or more barriers are coupled to the upper wall and the lower wall of the body to define the cavity.

5. The pollution control device of any one of claims 1 to 4, wherein the one or more barriers include more than one barrier, and each barrier is parallel with each other barrier.

6. The pollution control device of claim 5, wherein the barriers are equally spaced from each other.

7. The pollution control device of claim 5, wherein the barriers are unequally spaced from each other.

8. The pollution control device of any one of claims 1 to 7, wherein the flow disruptor extends from the barrier into one of the channels.

9. The pollution control device of claim 1, wherein the one or more barriers include two barriers, each barrier having a flow disruptor extending into a common channel.

10. The pollution control device of claim 1, wherein the one or more barriers include two barriers, each barrier having a plurality of a flow disruptors extending into a common channel, the plurality of flow disruptors being equally spaced from each other along a length the channel.

11. The pollution control device of any one of claims 1 to 10, wherein the light source is positioned on a top surface or a bottom surface of the body and directed towards the one or more channels.

12. The pollution control device any one of claims 1 to 10, wherein the light source is positioned on the one or more barriers and directed towards the one or more channels.

13. The pollution control device any one of claims 1 to 10, wherein the light source is positioned on the flow disruptor and directed towards the one or more channels.

14. The pollution control device any one of claims 1 to 10, wherein the light source is positioned at one of the first end and the second end and directed towards the one or more channels.

15. The pollution control device of any one of claims 1 to 10, wherein the barrier includes a plurality of flow disruptors dispersed along a length of the one or more channels and the light source is positioned between two of the plurality of flow disruptors.

16. The pollution control device of any one of claims 1 to 15, wherein the flow disruptor is coated with the photocatalytic composite material.

17. The pollution control device of any one of claims 1 to 15, wherein at least a portion of the one or more barriers is coated with the photocatalytic composite material.

18. The pollution control device of any one of claims 1 to 17, wherein the photocatalytic composite material is configured to remove NOₓ, volatile organic compounds, chlorinated solvents and/or ethylene (C₂H₄).

19. The pollution control device of claim 18, wherein the NOₓ is NO or NO₂.

20. The pollution control device of any one of claims 1 to 19, wherein at least one of the first end and the second end of the body, or the upper wall and lower wall of the body, or sidewalls of the body, is configured to couple to a neighboring pollution control device.

21. The pollution control device of any one of claims 1 to 20, wherein the body is sized and shaped to be housed in an air duct.

22. The pollution control device of any one of claims 1 to 21, further comprising a cooling system.

23. The pollution control device of claim 22, wherein the cooling system comprises a cooling apparatus disposed on or adjacent to an exterior surface of the body.

24. The pollution control device of claim 23, wherein the cooling apparatus includes a water pump and a water pipe fluidly coupled to the water pump, the water pump configured to move water through the water pipe across the exterior surface of the body.

25. The pollution control device of any one of claims 1 to 24, wherein the light source is an ultraviolet (UV) light source.

26. The pollution control device of any one of claims 1 to 24, wherein the light source is a visible light source.

27. A method of removing a gaseous pollutant from air, the method comprising:
activating a photocatalytic composite material disposed on a surface of a pollution control device by exposing the photocatalytic composite material to light, the activating of the photocatalytic composite material providing for the photocatalytic composite material to releasably adsorb the pollutants in the air; and
directing the air over the surface with the photocatalytic composite material for the pollutants in the air to adsorb to the photocatalytic composite material disposed on the surface.

28. The method of claim 27, further comprising:
exposing the photocatalytic composite material to light after the photocatalytic composite material has removed the pollutant adsorbed thereto to release the pollutant from the photocatalytic composite material or clean the surface of other contaminants.

29. The method of claim 28, further comprising:
directing the air over the surface to carry the pollutants out of the pollution control device.

30. The method of any one of claims 27 to 29, wherein the photocatalytic composite material is configured to remove NOₓ, volatile organic compounds, chlorinated solvents and/or ethylene (C₂H₄).

31. The method of any one of claims 27 to 30, wherein the pollution control device is the pollution control device of any one of claims 1 to 26.
